(19) United States
(12) Patent Application Publication
(10) Pub. No.: US 2009/0221908 A1
(43) Pub. Date: Sep. 3, 2009

(54) System and Method for Alignment of Instrumentation in Image-Guided Intervention

(76) Inventor: Neil David Glossop, Toronto (CA)

Correspondence Address: PILLSBURY WINTHROP SHAW PITTMAN, LLP, P.O. BOX 10500, MCLEAN, VA, 22102, US

(21) Appl. No.: 12/040,889

(22) Filed: Mar. 1, 2008

(51) Int. Cl. A61B 5/00

(52) U.S. Cl. 600/424; 382/128

(57) ABSTRACT

The invention provides systems and methods for aligning or guiding instruments during image-guided interventions. A volumetric medical scan (image data) of a patient may first be registered to patient space data regarding the patient obtained using a tracking device. An ultrasound simulator fitted with position indicating elements whose location is tracked by the tracking device is introduced to the surface of the anatomy of the patient and used to determine an imaginary ultrasound scan plane for the ultrasound simulator. This scan plane is used to reformat the image data so that the image data can be displayed to a user in a manner analogous to a handheld ultrasound transducer by re-slicing the image data according to the location and orientation of the ultrasound simulator. The location of an instrument fitted with position indicating elements tracked by the tracking device may be projected onto the re-sliced scan data.

### FIELD OF THE INVENTION

This invention relates to systems, methods, and instrumentation for facilitating accurate image-guided interventions using an ultrasound simulation device.

### BACKGROUND OF THE INVENTION

When performing image-guided interventions (IGI), it is often required to guide a needle or instrument to a location in the body. In many forms of IGI, preoperative or intraoperative scans are performed. In some instances preoperative scans include computerized tomography (CT), magnetic resonance (MR), positron emission tomography (PET), or single proton emission tomography (SPECT). These modalities tend to utilize volumetric data acquisition, providing full 3D data sets comprising multiple “slices” of data representing contiguous or overlapping cross sections through the data.

During an intervention, a physician may use a position sensing system (referred to herein as a “tracking device”) together with position indicating elements attached to individual instruments. The tracking device may be an optical camera array or an electromagnetic (EM) tracking device, a fiber optic device, a GPS sensor device, an instrumented mechanical arm or linkage, or other type of tracking device. In the case of optical camera tracking devices, the position indicating elements may be Light Emitting Diodes (LEDs) and in the case of EM tracking devices the position indicating elements may be sensor coils that receive or transmit an EM signal to or from the tracking device.

During image-guided interventions, physicians typically watch a screen onto which a representation of the location and trajectory of an instrument is displayed. Often the display can take the form of a 3D display in which the instrument is indicated in the screen as a graphic representation overlayed on a volume rendering, surface rendering, or other rendering of the patient anatomy. Another representation is an “axial-coronal-sagittal” reformat, where a crosshair shows the location of the tip of the instrument on an axial view of the data as well as coronal and sagittal views that have been fabricated from the slice stack. Another common display includes an “oblique reformat” view, in which the dataset from the preoperative scan is reformatted along a plane representing the instrument path. The instrument is shown within a cut representing the current and future trajectory of the device. Another representation is a so called targeting view or “flight path” view, in which a preplanned target is shown and graphic elements such as circles or other graphic elements representing the location and orientation of the instrument are aligned so that the device is presented in the correct view. Such views are similar to views available in airplane cockpits to assist in navigation. Many other representations are also possible.

In all of these cases, difficulties may be presented. The oblique reformat requires the physician to view multiple image displays at one time in order to properly line up the device. This can be mentally challenging and require great concentration. This format may also require a learning phase during the alignment of the needle due to disparate coordinate systems preventing the graphic representation of the device from moving “sensibly.” The flight path can sometimes be more intuitive, but requires a planning stage in which the physician preplans at least the target. Unless he also preplans the path, he may be unaware of the material which will be transversed during the insertion of the device, potentially leading to complications if a critical anatomical structure is breached along the path.

By contrast, many physicians are familiar with ultrasound devices and find the interface intuitive and instructive, since the transducer can be held and moved in a way so as to follow the instrument, to view anatomy and examine an instrument's path. By manipulating the transducer, views can be changed at will, unlike the aforementioned views that require manipulation of the computer's user interface. Unfortunately, this type of view is not available though existing image guided surgery systems.

For these reasons and others, current techniques may pose many difficulties.

### SUMMARY OF THE INVENTION

The invention addresses these and other difficulties in the art by providing a system, device, and methods for alignment and navigation of instrumentation during image-guided interventions. In some embodiments, a volumetric medical scan (image data) of a portion of the anatomy of a patient is loaded onto a computer that is connected to a tracking device capable of tracking the position and orientation of multiple position indicating elements in the tracking device's coordinate system. Patient space data regarding the anatomy of the patient may be obtained for example, using a registration device having one or more position indicating elements tracked by the tracking device. The patient space data is then registered to the volumetric image data.

A handheld ultrasound simulator fitted with one or more position indicating elements whose position and orientation (i.e., location within the coordinate system of the tracking device) are tracked by the tracking device is introduced to the surface or other portion of the anatomy of the patient. The position and orientation information of the ultrasound simulator is used to determine a simulated or imaginary ultrasound scan plane for the ultrasound simulator. This scan plane is used to reformat the image data so that the image data can be displayed to a user in a manner analogous to a handheld ultrasound transducer by re-slicing the image data according to the location and orientation of the ultrasound simulator. The location of an instrument fitted with one or more position sensors tracked by the tracking device may be projected onto the re-sliced scan data and the intersection of the trajectory of the tracked instrument and the imaginary scan plane may be calculated and displayed.

The various objects, features, and advantages of the invention will be apparent through the detailed description and the drawings attached hereto. It is also to be understood that the following detailed description is exemplary and not restrictive of the scope of the invention.

### BRIEF DESCRIPTION OF THE DRAWINGS

FIG. 1 illustrates an example of system for alignment of instrumentation during an image-guided intervention according to various embodiments of the invention.

FIG. 2 illustrates an ultrasound simulator according to various embodiments of the invention.

FIGS. 3A and 3B illustrate an ultrasound simulator, its associated scan plane and a tracked instrument according to various embodiments of the invention.

FIG. 4 illustrates a process for alignment of instrumentation during an image-guided intervention according to various embodiments of the invention.

FIG. 5 illustrates an ultrasound simulator, a body, and a tracked instrument according to various embodiments of the invention.

FIG. 6A illustrates a reformatted image according to various embodiments of the invention.

FIG. 6B illustrates a coordinate system including an actual path of a tracked instrument through a scan plane of an ultrasound simulator according to various embodiments of the invention.

FIG. 7 illustrates a process for alignment of instrumentation on a training apparatus according to various embodiments of the invention.

### DETAILED DESCRIPTION OF PREFERRED EMBODIMENTS

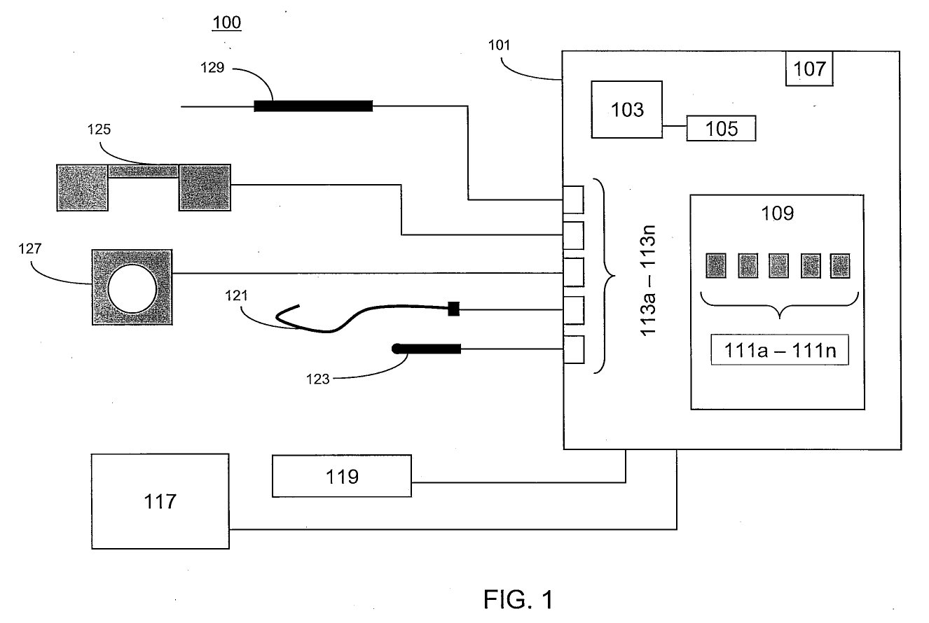

FIG. 1 illustrates a system 100, which is an example of a system for alignment and navigation of instrumentation during an image-guided intervention. System 100 may include a computer element 101, a registration device 121, an ultrasound simulator 123, a tracking device 125, an imaging device 127, a tracked instrument 129, and/or other elements.

Computer element 101 may include a processor 103, a memory device 105, a power source 107, a control application 109, one or more software modules 111a-111n, one or more inputs/outputs 113a-113n, a display device 117, a user input device 119, and/or other elements.

Computer element 101 may be or include one or more servers, personal computers, laptop computers, or other computer devices. In some embodiments, computer element 101 may receive, send, store, and/or manipulate data necessary to perform any of the processes, calculations, image formatting, image display, or operations described herein. In some embodiments, computer element 101 may also perform any processes, calculations, or operations necessary for the function of the devices, elements, instruments, or apparatus described herein.

In some embodiments, computer element 101 may host a control application 109. Control application 109 may comprise a computer application which may enable one or more software modules 111a-111n. One or more software modules 111a-111n enable processor 103 to receive (e.g., via a data reception module), send, and/or manipulate image data in the coordinate system of an imaging modality (including volumetric image data) regarding the anatomy of a patient, one or more objects (e.g., a phantom object or representative anatomical model) and/or other image data. This image data may be stored in memory device 105 or other data storage location. In some embodiments, one or more software modules 111a-111n may also enable processor 103 to receive (e.g., via the data reception module), send, and/or manipulate data regarding the location, position, orientation, and/or coordinates of one or more position indicating elements (e.g., sensor coils or other position indicating elements). This data may be stored in memory device 105 or other data storage location.

In some embodiments, one or more software modules 111a-111n such as, for example, a registration module may also enable processor 103 to calculate one or more registration transformations, perform registration (or mapping) of coordinates from two or more coordinate systems according to the one or more transformation calculations.

In some embodiments, one or more software modules 111a-111n such as, for example, a display module, may enable processor 103 to produce, format, and/or reformat one or more images from image data, position/orientation/location data, and/or other data. In some embodiments, images produced from image data, position/orientation/location data, other data, or any combination thereof may be displayed on display device 117. In some embodiments, one or more software modules 111a-111n such as, for example, the display module, may enable the generation and display of images of the anatomy of the patient or an object (e.g., a phantom object or representative anatomical model) with the position and/or orientation of a tracked instrument superimposed thereon in real time (such that motion of the tracked instrument within the anatomy of the patient is indicated on the superimposed images) for use in an image-guided procedure. In some embodiments, the images on which the tracked instrument are displayed may be formatted to specifically display any anatomy or portion of a device intersected by an imaginary scan plane of an ultrasound simulator and/or any number of perspective views of or involving this imaginary scan plane. For example, if the imaginary scan plane is aligned so that it extends into the patient to from a cut extending from the anterior of the patient through to the posterior, the view displayed to a user may appear as an axial cut through the patient. Similarly, if the imaginary scan plane was aligned longitudinally along the patient's body, a sagittal cut may be displayed. Any oblique orientation of the imaginary scan plane may yield a view of an oblique cut through the patient.

In some embodiments, system 100 may include a registration device 121 connected to computer element 101 via an input/output 113. Registration device 121 may provide position and or orientation data regarding one or more points or areas within or on an anatomical region of a patient. The registration device may otherwise enable registration of the anatomical region the patient, (including soft tissues and/or deformable bodies) and may include one or more position indicating elements (e.g., sensor coils) whose position and/or orientation are trackable by tracking device 125 in the coordinate system of tracking device 125.

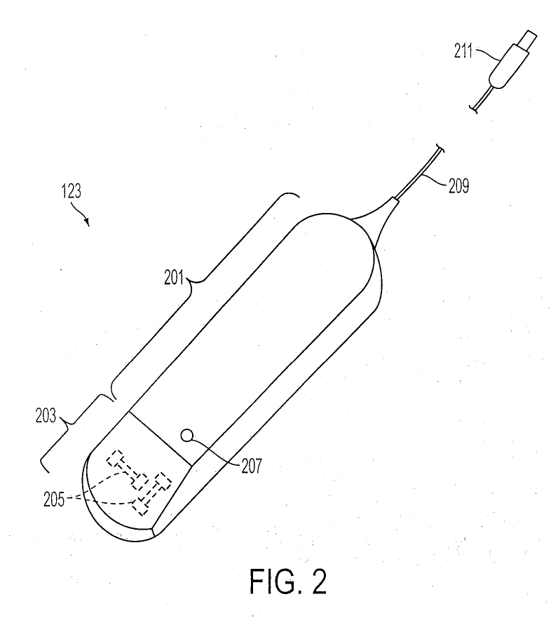

In some embodiments, system 100 may include an ultrasound simulator 123. FIG. 2 illustrates an example of ultrasound simulator 123, which may be representative of a conventional ultrasound hand-piece. In some embodiments, ultrasound simulator 123 may include a handle portion 201, a front portion 203, one or more position indicating elements 205, one or more LEDs 207, a cable 209, a connector 211, and/or other elements.

The one or more position indicating elements 205 may enable the determination of a position (for example, position in Cartesian, spherical space, or other coordinate system) and orientation (for example, the roll, pitch, and yaw) of ultrasound simulator 123 in a coordinate system of tracking device 125. As such, ultrasound simulator 123 may be connected to tracking device 125 and/or computer element 101 such that position and orientation information regarding the one or more position indicating elements 205 is communicated to computing element 101.

In some embodiments, ultrasound simulator 123 may be tracked in 6 degrees of freedom using the one or more position indicating elements 205. In another embodiment, it may be tracked in fewer degrees of freedom. While FIG. 2 illustrates two position indicating elements 205, in some embodiments, only one position indicating element may be used. For example, if a single position indicating element 205 were capable of providing information regarding 6 degrees of freedom and information regarding 6 degrees of freedom were desired, only a single position indicating element 205 may be used. However, if position indicating elements 205 capable of determining less than 6 degrees of freedom were used and information regarding 6 degrees of freedom were desired, two or more position indicating elements 205 may be used. In some embodiments, the one or more position indicating elements 205 may be embedded or integrated into ultrasound simulator 123 (hence they are illustrated using dashed lines in FIG. 2). However, in some embodiments, they may be mounted on the surface of ultrasound simulator 123 or located elsewhere on or in ultrasound simulator 123 such that they are rigidly associated with ultrasound simulator 123.

Cable 209 and connector 211 may connect the one or more position indicating elements 205, LEDs 207, and/or other elements of ultrasound simulator 129 to tracking device 125, computer element 101, and/or a power source. In some embodiments, data from position indicating elements 205 may be otherwise exchanged (e.g., wirelessly) with tracking device 125 or computer element 101.

In some embodiments, ultrasound simulator 123 may be mechanically attached to additional elements such, for example, a mechanical digitizing linkage type of tracking device that enables measurement of the location and orientation of ultrasound simulator 123. The mechanical digitizing linkage tracking device may be used in place of or in addition to tracking device 125 and one or more position indicating elements 205 to obtain position and orientation information regarding ultrasound simulator 123.

In some embodiments, ultrasound simulator 123 may include additional emitter or sensor elements such as, for example, temperature sensors, pressure sensors, optical emitters and sensors, ultrasound emitters and sensors, microphones, electromagnetic emitters and receivers, microwave sensors or emitters, or other elements that perform therapeutic, diagnostic, or other functions. It may also include visual indication elements such as visible LEDs (e.g., LED 207), LCD displays, video displays or output or input devices such as buttons, switches or keyboards.

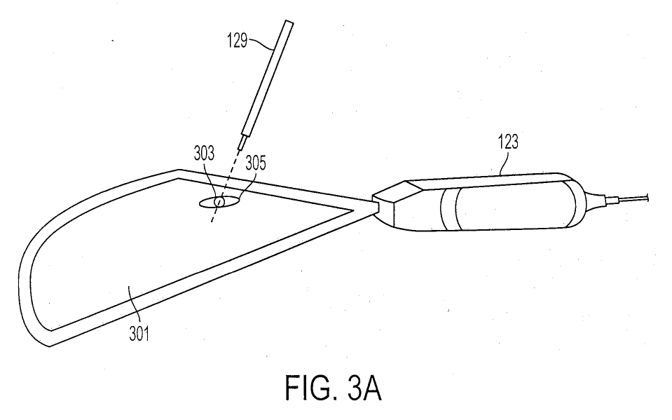

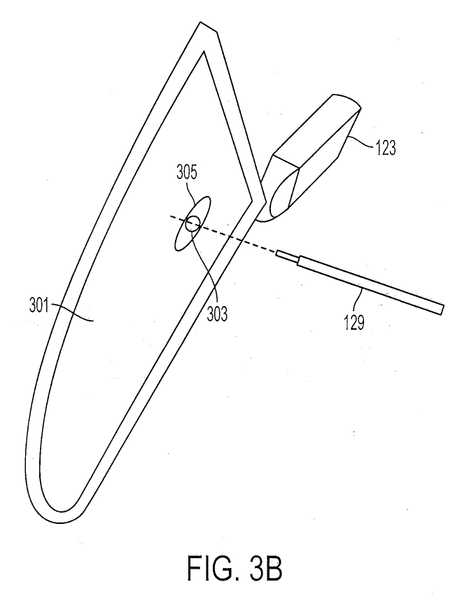

Ultrasound simulator 123 may be calibrated so that the location and orientation of front portion 203 (which contacts a patient) is known relative to the coordinate system of position indicating elements 205 and therefore tracking system 125. In particular, ultrasound simulator 123 may be calibrated so that a plane representing the “scan plane” of the simulator that is analogous to an ultrasound transducer scan plane is known. Such an “imaginary” or “simulated” scan plane may be orientated extending out from front portion 203 of ultrasound simulator 123. See for example, scan plane 301 as illustrated in FIGS. 3A and 3B.

In some embodiments, system 100 may also include a tracking device 125. In one embodiment, tracking device 125 may be operatively connected to computer element 101 via an input/output 113. In some embodiments, tracking device 125 need not be operatively connected to computer element 101, but data may be sent and received between tracking device 125 and computer element 101. Tracking device 125 may include an electromagnetic tracking device, global positioning system (GPS) enabled tracking device, an ultrasonic tracking device, a fiber-optic tracking device, an optical tracking device, radar tracking device, or other type of tracking device. Tracking device 125 may be used to obtain data regarding the three-dimensional location, position, orientation, coordinates, and/or other information regarding one or more position indicating elements (including position indicating elements 205 of ultrasound simulator 123 and any position indicating elements located on registration device 121, tracked instrument 129, or other elements used with system 100). In some embodiments, tracking device 125 may provide this data/information to computer element 101.

In some embodiments, system 100 may include an imaging device 127. In one embodiment, data may be sent and received between imaging device 127 and computer element 101. This data may be sent and received via an operative connection, a network connection, a wireless connection, through one or more floppy discs, CDs DVDs or through other data transfer methods. Imaging device 127 may be used to obtain image data (including volumetric or three dimensional image data) or other data necessary for enabling the apparatus and processes described herein. Imaging device 127 may provide this data to computer element 101, where it may be stored. In some embodiments, a system for aligning instrumentation during an image-guided intervention need not include an imaging device 127, rather ultrasound simulator 123 may be connected to a computer element 101 to which data regarding scans from an imaging device 127 previously is loaded.

Imaging device 127 may include one or more of a computerized tomography (CT) device, positron emission tomography (PET) device, magnetic resonance (MR) device, single photon emission computerized tomography (SPECT) device, 3D ultrasound device or other medical imaging device that provides scans (image data) representing a volume of image data (i.e., volumetric image data). In some embodiments the scans or image data may be stored in the memory 105 (such as, for example, RAM, flash memory, hard disk, CD, DVD, or other storage devices) of computer element 101. The image data may be capable of being manipulated (e.g., by a display module) so as to enable the volume of data to be mathematically reformatted in such a way as to display a representation of the data as it would appear if it were cut, sliced, and/or viewed in any orientation.

System 100 may also include one or more tracked instruments 129. A tracked instrument 129 may include therapy devices or diagnostic devices that include one or more positions indicating elements whose position and orientation can be tracked by tracking device 125 simultaneously to ultrasound simulator 123. For example, in some embodiments, a tracked instrument 129 may include tracked needles, endoscopes, probes, scalpels, aspiration devices, or other devices. Other examples include the devices disclosed in US Patent Publication No. 20060173291 (U.S. patent application Ser. No. 11/333,364), 20070232882 (U.S. patent application Ser. No. 11/694,280), and U.S. Patent Publication No. 20070032723 (U.S. patent application Ser. No. 11/471,604), each of which are hereby incorporated by reference herein in their entirety.

In some embodiments, one or more tracked instruments 129, registration devices 121, ultrasound simulators 123, and/or other elements or devices described herein may be interchangeably “plugged into” one or more inputs/outputs 113a-113n. In some embodiments, various software, hardware, and/or firmware may be included in system 100, which may enable various imaging, referencing, registration, navigation, diagnostic, therapeutic, or other instruments to be used interchangeably with system 100. In some embodiments, the software, firmware, and/or other computer code necessary to utilize various elements described herein such as, for example, display device 117, user input 119, registration device 121, ultrasound simulator 123, tracking device 125, imaging device 127, tracked instrument 129 and/or other device or element, may be provided by one or more of modules 111a-111n.

Those having skill in the art will appreciate that the invention described herein may work with various system configurations. Accordingly, more or less of the aforementioned system components may be used and/or combined in various embodiments. It should also be understood that various software modules 111a-111n (including a data reception module, a registration module, and a display module) and control application 109 that are used to accomplish the functionalities described herein may be maintained on one or more of the components of system recited herein, as necessary, including those within individual medical tools or devices. In other embodiments, as would be appreciated, the functionalities described herein may be implemented in various combinations of hardware and/or firmware, in addition to, or instead of, software.

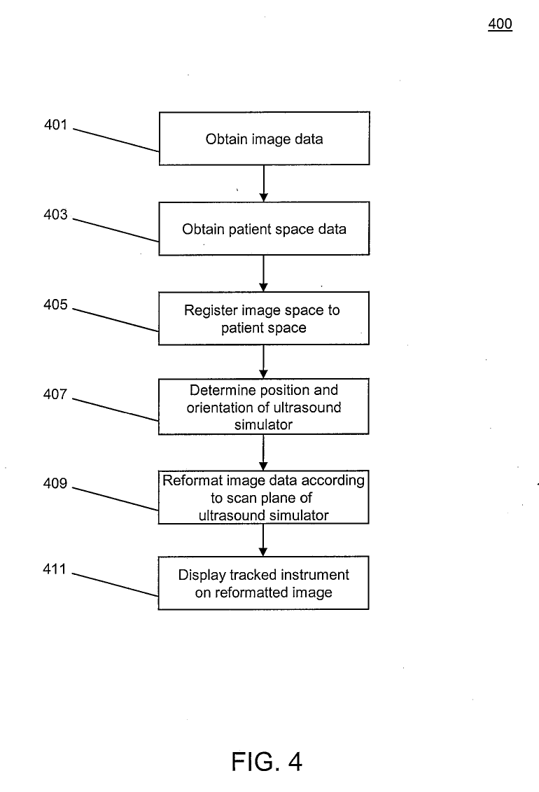

FIG. 4 illustrates a process 400, which is an example of a process for aligning and/or guiding instrumentation during an image-guided intervention according to various embodiments of the invention. Process 400 includes an operation 401, wherein one or more volumetric images (image data) of all or a portion of the anatomy of a patient are acquired by an imaging device (e.g., imaging device 127). As mentioned above, the image data may comprise or include a volume of data that can be mathematically reformatted in such a way as to display a representation of the data as it would appear if it were cut, sliced, and/or viewed in any orientation. The image data may then be communicated to and loaded onto computer element 101. For purposes of registration of the anatomy of the patient (or a region thereof) or other purposes, the image data may be considered or referred to as “image space data.”

In some embodiments, prior to obtaining the image data, the patient may be outfitted with one or more registration aids in anticipation of a registration operation. In some embodiments, the registration aids may include active or passive fiducial markers as known in the art. In some embodiments, no such registration aids are required.

In an operation 403, “patient space” data regarding the portion of the anatomy of the patient whereupon the image-guided intervention is to be performed may be obtained. For example, the patient space data may be obtained using a registration device having one or more position indicating elements (e.g., registration device 121) whose position and orientation are tracked by a tracking system (e.g., tracking system 125). The patient space data may be obtained in any number of ways depending on the surgical environment, surgical application, or other factors. For example, registration device 121 may be placed within the anatomy of the patient and information regarding the positions and/or orientation of the one or more position indicating elements of registration device 121 may be sampled by tracking device 125 and communicated to computer element 101. Information regarding obtaining patient space data and other information regarding registration of image space data to patient space data can be found in U.S. Patent Publication No. 20050182319 (U.S. patent application Ser. No. 11/059,336), which is hereby incorporated herein by reference in its entirety.

In an operation 405, the image space data may be registered to the patient space data. Registering the position of an anatomical object or region in a patient coordinate system (“patient space”) to views of the anatomical object in an image coordinate system (“image space”) may be performed using various methods such as, for example, point registration, path registration, surface registration, intrinsic registration or other techniques. Additional information relating to registration techniques can be found in U.S. Patent Publication No. 20050182319 (U.S. patent application Ser. No. 11/059,336) and U.S. Patent Publication No. 20060173269 (U.S. patent application Ser. No. 11/271,899), both of which are hereby incorporated by reference herein in their entirety. In some embodiments, the registration of operation 405 may be performed after scanning/imaging of operation 401 so that the patient's coordinate system is known in the coordinate system that the images were acquired in and vice versa.

Once registration has been performed, it may be possible to represent any tracked tool or instrument (e.g., tracked instrument 129) positioned in the coordinate system of the tracking device used to obtain the patient space data (e.g., tracking device 125) and thus the patient, in the coordinate system of the preoperative scan (e.g., overlayed or superimposed or otherwise integrated onto a graphical representation of the image data obtained in operation 401). As ultrasound simulator 123 is also tracked by the tracking device (due to being equipped with one or more position indicating elements 205), the location and orientation of ultrasound simulator 123 may also be determined relative to the coordinate system of the preoperative scan in an operation 407 and displayed as a graphical representation on the preoperative image data. Additionally, in operation 407, the location of scan plane 301 of ultrasound simulator 123 may be determined relative to the coordinate system of the preoperative scan and displayed on the preoperative image data.

In an operation 409, the position and orientation of ultrasound simulator 123 may be used to reformat the image data so that a view of the image data coincident to scan plane 301 of ultrasound transducer 123 can be displayed. The reformatting of the volumetric image data may include “re-slicing” the image data along the plane defined by scan plane 301 of ultrasound simulator 123. This may involve determining the intersection plane of scan plane 301 with the image data and displaying the intersection of scan plane 301 with the volume images acquired in operation 401. As ultrasound simulator 123 is moved over the patient, the view displayed to a user (e.g., via display 117) may be reformatted in real-time according to the position and orientation of ultrasound simulator 301 to provide a view, using the image data, of scan plane 301 of ultrasound simulator 123. In some embodiments, an algorithm may be used to reformat the image data to simulate the data of an ultrasound, so to create an oblique reformat along the scan plane of the simulator that appears similar to an ultrasound view.

In an operation 411, the location of additional instrumentation (e.g., tracked instrument 129) may be projected onto or otherwise integrated into the displayed image data (e.g., the reformatted view of the scan plane). In some embodiments, the location and orientation of tracked instrument 129 may be simultaneously displayed on the dataset that has been reformatted as determined by the location and orientation of ultrasound simulator 129. Since the reformatted dataset may generally be oriented in a different plane than tracked instrument 129, a “projection” of the instrument may be displayed on the slice relative to any anatomy or other elements intersecting the scan plane 301.

In some embodiments, the location that tracked instrument 129 crosses scan plane 301 of ultrasound simulator 123 may be indicated on the slice. FIGS. 3A and 3B illustrate that the crossing of the additional instrumentation (tracked instrument 129) with the scan plane may be indicated as an intersection point 303 for a substantially linear device such as a needle or catheter. To indicate an approximate crossing point, a circle 305 may be used to represent the crossing point within an amount of error. In some embodiments, the crossing may be indicated as a line for a substantially planar tracked instrument such as, for example, a blade. To indicate an approximate crossing line, a rectangle may be used to represent the crossing within an amount of error. In some embodiments, for a volumetric tracked instrument such as, for example, a deployable radiofrequency ablation device, the crossing may be indicated as the shape formed by the intersection of the device with the scan plane of the simulator. An enlarged intersection region may be used to indicate some degree of error in the system. In general, the intersection of scan plane 301 of ultrasound simulator 123 and tracked instrument 129 will change as tracked instrument 129 and/or ultrasound simulator 123 (and thus scan plane 301) are moved.

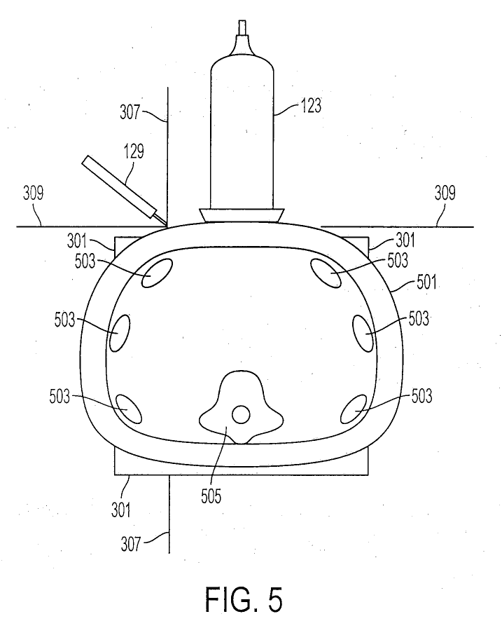

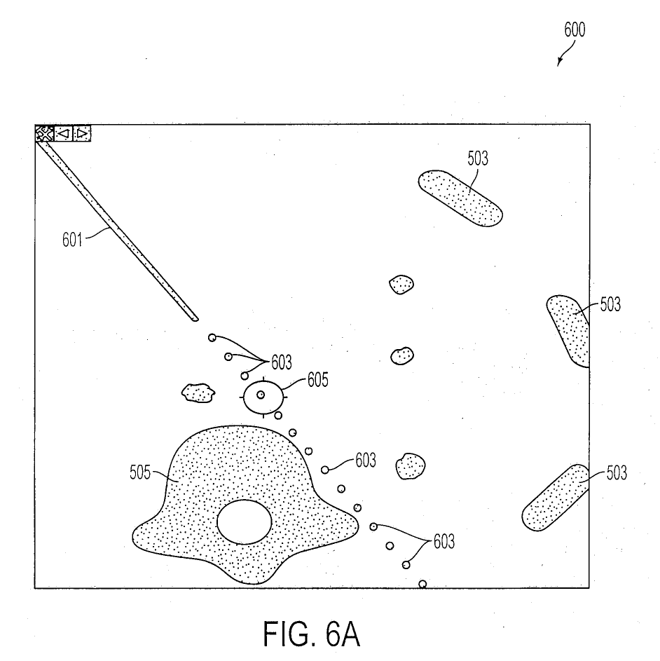

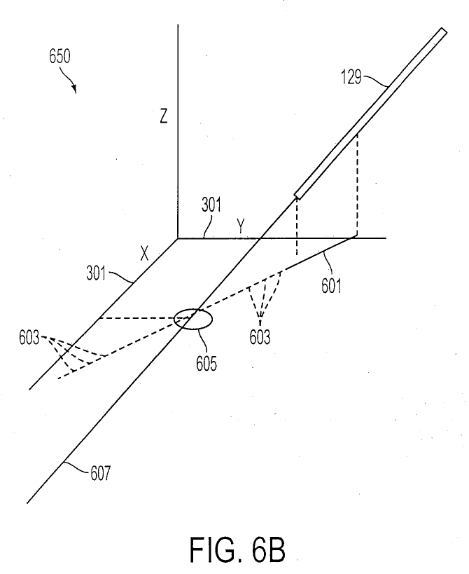

FIG. 5 illustrates ultrasound simulator 123 in contact with body 501 (which may be or simulate an anatomy of a patient), having minor internal features 503 and major internal feature 505. Scan plane 301 of ultrasound simulator 123 is also shown, as well as tracked instrument 129 and crosshairs 507 and 509, which pinpoint the tip of tracked instrument 129. FIG. 6A illustrates an image 600 that is an oblique reformatted view of scan plane 301 created using reformatted volumetric image data regarding body 501 and position and orientation data regarding ultrasound simulator 123. The volumetric image data is reformatted according to the position and orientation information of ultrasound simulator 123 to enable image 600, which is a view of a scan plane of ultrasound simulator 123 similarly positioned to the position shown in FIG. 5. However, unlike FIG. 5, wherein the tip of tracked instrument 129 is indicated as outside of body 501, image 600 illustrates that tracked instrument 129 has been partly inserted into body as evidenced by the solid indicator 601, which indicates the space occupied by tracked instrument 129 as projected onto the scan plane of the ultrasound simulator. A predicted path of tracked instrument 129 may also be provided, likewise projected onto the scan plane. Image 600 illustrates dots or marks 603, indicating the predicted path of tracked instrument 129. Circle 605 indicates the calculated area where tracked instrument 129 will cross the scan plane of ultrasound simulator 123 on its current trajectory.

FIG. 6B illustrates a coordinate system 650, wherein the scan plane 301 of ultrasound simulator 123 is represented by the X and Y axes. As illustrated, the plane of the trajectory of tracked instrument 129 is not in the same plane as scan plane 301. However, indicator 601 is projected onto scan plane 301 (and thus image 600 of FIG. 6A) for the benefit of the user. Similarly, the predicted path of tracked instrument, indicated as line 607 may also be projected onto the image (e.g., as dots 603 [or dashes 603 in FIG. 6B]). As stated above, the predicted point where tracked instrument 129 will intersect scan plane 301 is indicated on the image by circle 605.

As tracked instrument 129 is moved, indicator 601, dots 603, and circle 605 are adjusted accordingly on image 600. If ultrasound simulator 123 is moved, then the scan will be reformatted or “sliced” differently to show an image relative to the new scan plane of ultrasound simulator 123. Depending on the orientation of the ultrasound simulator, the view of FIG. 6 will be different. If the trajectory of the instrument 129 is substantially in the same plane as the scan plane of the ultrasound simulator, the instrument will no longer cross the scan plane, since it is already in it. Also, what was previously a “projection” of the instrument path in the scan plane would in fact represent the actual predicted path of the instrument. The physician may move the ultrasound simulator handle to view many different cut planes through the anatomy and see the predicted location that the instrument's path will cross or does cross that plane.

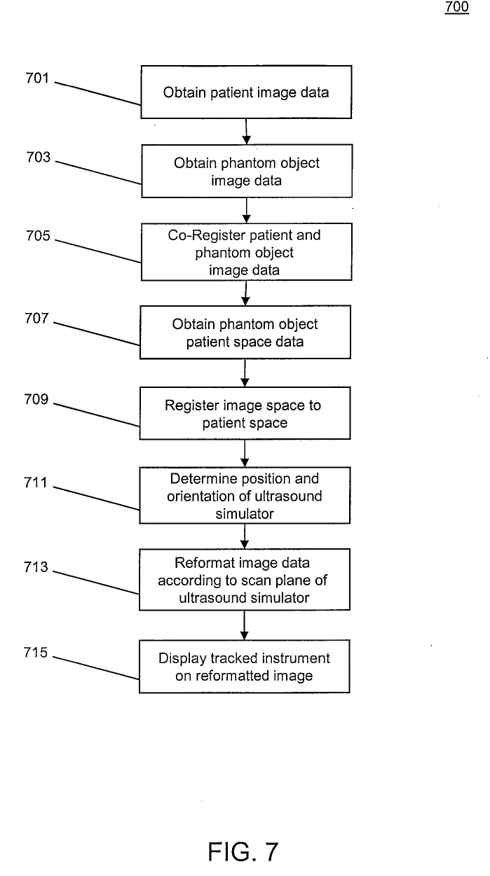

In some embodiments, the invention includes a system and process for training users (e.g., physicians) to utilize ultrasound simulator 123 (and/or other system elements) for alignment of instrumentation during an image-guided intervention. FIG. 7 illustrates a process 700, which is an example of a process for training users to utilize ultrasound simulator 123 for alignment of instrumentation during an image-guided intervention. Process 700 may be performed using part or all of the system components of system 100. Process 700 may also utilize a surrogate patient anatomy element or “phantom object” (also referred to as a “phantom”) upon which training is performed (rather than the anatomy of a patient) to simulate or substitute for the actual anatomy of the patient. In some embodiments, the phantom object may be constructed of an inert material such as, for example, rubber, gel, plastic, or other material. In some embodiments, the shape of the phantom object may be anthropomorphic. In some embodiments, the phantom object may be non-anthropomorphic. In some embodiments, the phantom object any include features that are representative of a real patient including simulated bones, simulated tumors, or other features.

Process 700 includes an operation 701, wherein, similar to operation 401, image data of an actual patient may be obtained. In an operation 703, image data regarding the phantom object may also be obtained. In some embodiments, at least one of the image data sets (i.e., actual patient image data or phantom object image data) may be volumetric image data. In an operation 705, the patient image data may be co-registered to the phantom object image data. In embodiments wherein only one type of image data is used (e.g., only actual patient image data or phantom object image data, thus there may be no co-registration operation 705), the image data used may be volumetric image data. In an operation 707, patient space data regarding the phantom object may be obtained. This patient space data may be obtained using a tracked probe or other tracked device such as, for example, registration device 121 in a manner similar to that described herein regarding operation 403.

In an operation 709, the co-registered image data (patient and phantom object image data) may be registered to the patient space data from the phantom object. In instances where phantom object image data is not obtained, the image data from the patient may be registered to the patient space data from the phantom object. In other embodiments, training may be performed using only image space data regarding the phantom object that is registered to patient space data regarding the phantom object. Registration may be carried out by any of the aforementioned methods or other methods of registration.

In an operation 711, an ultrasound simulator that is tracked by the tracking device used to obtain the phantom object patient space data (e.g., ultrasound simulator 123) may be introduced to the surface or other portion of the phantom object and the position and orientation of the ultrasound simulator may be determined. Additionally, the intersection of the scan plane of the ultrasound simulator and the image data may be determined.

In an operation 713, the image data (e.g., co-registered patient and phantom object data, patient image data only, or phantom object image data only), may then be reformatted to display a view of the “scan plane” of the ultrasound simulator (e.g., at an oblique view). In an operation 715, an instrument tracked by the tracking device used to obtain the patient space data of the phantom object and track the ultrasound simulator (e.g., tracked instrument 129), may be introduced to the phantom object and displayed on the reformatted view of the image data. As the tracked instrument moves, its display on the reformatted image data may be moved. As the ultrasound simulator is moved, the image data is reformatted or “re-sliced” (including a new determination of where the new scan plane intersects the image data) to show a view of the new scan plane of the ultrasound simulator and thus the tracked instrument relative to the portions of the phantom object intersected by the scan plane.

In this manner, a user may be trained to navigate any number of tracked instruments while manipulating the ultrasound simulator around the phantom object. Depending on the design of the phantom object, the user may train for countless specific circumstances (e.g., for specific types of anatomy specific targets, or other scenarios, as reflected by the features of the phantom object).

For example, in some embodiments, the phantom object may include a “pre-selection apparatus.” The pre-selection apparatus may include one or more elements that enable an operator to pre-select a three dimensional location (“target point”) in the phantom object that may act as a target for training purposes. For example, the pre-selection apparatus may be used to designate a “proxy tumor” in a phantom object for the purposes of training a user. The systems and methods of the invention may be then be used by a trainee to help locate the proxy tumor. For example, a needle or crossing light beams may be used to demarcate the proxy tumor. In one example, a real patient's image data may be co-registered with an inert gel phantom object that enables the trainee to insert tracked therapy devices such as needles into it. In some embodiments, the phantom object may include actual (physical) proxy tumors such as blobs of different colored or different density of gel. In some embodiments, a tracked needle or therapy device is directed using the systems and methods of the invention to the location of the proxy tumor within the phantom object. To score the trainee, the proximity of the tracked device may be compared to the position of the proxy tumor.

In some embodiments, the pre-selection apparatus may be a mechanical device such as a stereotactic framework for positioning a needle or device to a particular location. The framework may enable the location of the needle or device to be adjusted by dials, knobs, motors, or other elements included in the framework. As discussed above, in some embodiments, the tip of the needle or device may act as a lesion or other feature for training in order to co-locate a tracked instrument to the same location using the systems and method of the invention.

In some embodiments, the pre-selection apparatus may include elements for optically designating an interior target point in a transparent or translucent phantom object, for example, by using two or more lasers to intersect on a location. In some embodiments, the lasers may be positioned using framework and/or a motor system.

While the methods processes described herein have been described as method and processes for aligning and navigating instrumentation during image guided surgery, the invention may also provide systems and methods (or processes) for visualizing or displaying a portion of the anatomy of a patient using an ultrasound simulator (e.g., ultrasound simulator 123) and/or other system elements described herein.

In some embodiments, the invention includes a computer readable medium having computer readable instructions thereon for performing the various features and functions described herein, including one or more of the operations described in process 400 and 700, and/or other operations, features, or functions described herein.

It should be understood by those having skill in the art that while the operations of the methods and processes described herein have been presented in a certain order, that the invention may be practiced by performing the operations, features, and/or functions described herein in various orders. Furthermore, in some embodiments, more or less of the operations, features, and/or functions described herein may be used.

Other embodiments, uses and advantages of the invention will be apparent to those skilled in the art from consideration of the specification and practice of the invention disclosed herein. The specification should be considered exemplary only, and the scope of the invention is accordingly intended to be limited only by the following claims.

What is claimed is:

1. A method for aligning or guiding a tracked instrument during an image-guided intervention, the method comprising:
registering volumetric image data of an anatomy of a patient to patient space data of the anatomy of the patient, wherein the patient space data is obtained using a tracking device;
determining, in the coordinate system of the tracking device, location information regarding one or more position indicating elements rigidly associated with an ultrasound simulator using the tracking device;
determining an imaginary scan plane of an ultrasound simulator in a the coordinate system of the tracking device;
determining an intersection of the imaginary scan plane of the ultrasound simulator through the volumetric image data of the anatomy of the patient using the location information of the one or more position indicating elements;
formatting at least a portion of the volumetric image data into a display of the anatomy of the patient intersected by the imaginary scan plane; and
displaying a location of a tracked instrument on the display, the tracked instrument including one or more position indicating elements tracked by the tracking device.

2. The method of claim 1, wherein the volumetric image data of the anatomy of the patient is obtained by one or more of a computerized tomography imaging modality, a magnetic resonance imaging modality, a positron emission tomography imaging modality, or a single proton emission tomography imaging modality.

3. The method of claim 1, further comprising displaying a projected path of the tracked instrument on the display.

4. The method of claim 1, further comprising updating the display to reflect movement by the tracked instrument.

5. The method of claim 1, further comprising:
moving the ultrasound simulator such that imaginary scan plane intersects a different portion of the anatomy of the patient;
formatting at least a portion of the volumetric image data into a second display of the different portion of the anatomy of the patient intersected by the imaginary scan plane; and
displaying the location of the tracked instrument on the second display.

6. The method of claim 1, wherein the display of the anatomy of the patient comprises an oblique angle view of the anatomy of the patient created by the intersection of the imaginary scan plane with the volumetric image data.

7. The method of claim 1, wherein location information includes position and orientation information.

8. A system for aligning or guiding a tracked instrument during an image-guided intervention, the system comprising:
a tracking device that obtains patient space data regarding an anatomy of a patient, determines location information regarding one or more position indicating elements rigidly associated with an ultrasound simulator, and tracks one or more position indicating elements associated with a tracked instrument;
a registration module that registers volumetric image data of the anatomy of a patient to the patient space data of the anatomy of the patient; and
a display module that determines an intersection of an imaginary scan plane from a front portion of the ultrasound simulator through the volumetric image data of anatomy of the patient using the location information of the position indicating elements and formats at least a portion of the volumetric image data into a display of the anatomy of the patient intersected by the imaginary scan plane, wherein the display indicates a location of the tracked instrument relative to the anatomy of the patient intersected by the imaginary scan plane.

9. The system of claim 8, wherein the display includes a projected path of the tracked instrument on the display.

10. The system of claim 8, wherein the display module updates the display to reflect movement by the tracked instrument.

11. The system of claim 8, wherein the display module formats at least a portion of the volumetric image data into a second display of a different portion of the anatomy of the patient intersected by the imaginary scan plane when the ultrasound simulator is moved such that imaginary scan plane intersects the different portion of the anatomy of the patient, wherein the second display includes the location of the tracked instrument relative to the different portion of the anatomy of the patient.

12. The system of claim 8, the display of the anatomy of the patient comprises an oblique angle view of the anatomy of the patient created by the intersection of the imaginary scan plane and the volumetric image data.

13. A method for training users to align instrumentation during image guided surgery, the method comprising:
registering volumetric image data of an anatomy of a patient to patient space data of a phantom object representing the anatomy of the patient, wherein the patient space data is obtained using a tracking device;
determining, in the coordinate system of the tracking device, location information regarding one or more position indicating elements rigidly associated with an ultrasound simulator using the tracking device;
determining an imaginary scan plane of the ultrasound simulator in the coordinate system of the tracking device;
determining an intersection of the imaginary scan plane of the ultrasound simulator through the volumetric image data of the phantom object using the location information of the one or more position indicating elements;
formatting at least a portion of the volumetric image data into a display of a portion of the phantom object intersected by the imaginary scan plane; and
displaying a location of a tracked instrument relative to the portion of the phantom object intersected by the imaginary scan plane on the display, the tracked instrument including one or more position indicating elements tracked by the tracking device.

14. The method of claim 13, further comprising displaying a projected path of the tracked instrument on the display.

15. The method of claim 13, wherein the phantom object includes a target therein.

16. The method of claim 15, wherein the phantom object is made of a translucent material, and wherein the target is provided by an intersection of two or more energy beams projected into the phantom object.

17. The method of claim 15, wherein the target is provided by a tip of a needle positioned within the phantom object.

18. The method of claim 15, wherein the target is provided by a portion of the target device having one or more of a differentiating color or a differentiating density.

19. The method of claim 13, further comprising co-registering the volumetric image data of the anatomy of the patient to volumetric image data of the phantom object prior to registering the volumetric image data of the anatomy of the patient to the patient space data of the phantom object.

20. A system for training users to align instrumentation during an image-guided intervention, the system comprising:
a phantom object that simulates a portion of an anatomy a patient;
a tracking device that obtains patient space data regarding the phantom object, determines location information regarding one or more position indicating elements rigidly associated with an ultrasound simulator, and tracks one or more position indicating elements associated with a tracked instrument;
a registration module that registers volumetric image data of the anatomy of a patient to the patient space data of the phantom object; and
a display module that determines an intersection of an imaginary scan plane from a front portion of the ultrasound simulator through the volumetric image data of the phantom object using the location information of the one or more position indicating elements and formats at least a portion of the volumetric image data into a display of the phantom object intersected by the imaginary scan plane, wherein the display indicates a location of the tracked instrument relative to the portion of the phantom object intersected by the imaginary scan plane.

21. The system of claim 20, wherein the display further includes a projected path of the tracked instrument.

22. The system of claim 20, wherein the phantom object is made of a translucent material and includes a target therein that is provided by an intersection of two or more energy beams projected into the phantom object.

23. The system of claim 20, wherein phantom object includes a target that is provided by a tip of a needle positioned within the phantom object.

24. The system of claim 20, wherein the phantom object includes a target that is provided by a portion of the target device having one or more of a differentiating color or a differentiating density.

25. A method for displaying a portion of an anatomy of a patient, the method comprising:
registering volumetric image data of an anatomy of a patient to patient space data of the anatomy of the patient, wherein the patient space data is obtained using a tracking device;
determining, in a coordinate system of the tracking device, location information regarding one or more position indicating elements rigidly associated with an ultrasound simulator using the tracking device;
determining an imaginary scan plane of the ultrasound simulator in the coordinate system of the tracking device;
determining an intersection of the imaginary scan plane of the ultrasound simulator through the volumetric image data of the anatomy of the patient using the location information of the one or more position indicating elements; and
formatting at least a portion of the volumetric image data into a display of the anatomy of the patient intersected by the imaginary scan plane.
